(19) United States
(12) Patent Application Publication
(10) Pub. No.: US 2015/0238700 A1
(43) Pub. Date: Aug. 27, 2015

(54) Method for Assembling a Drug Delivery Device

(71) Applicant: Sanofi-Aventis Deutschland GmbH, Frankfurt am Main (DE)

(72) Inventors: Michael Jugl, Frankfurt am Main (DE); Axel Teucher, Frankfurt am Main (DE)

(21) Appl. No.: 14/421,123

(22) PCT Filed: Aug. 15, 2013
(86) PCT No.: PCT/EP2013/067059

§ 371 Date: Feb. 11, 2015

(30) Foreign Application Priority Data
Aug. 20, 2012 (EP) 12180960.2

(51) Int. Cl. A61M 5/315

(52) U.S. Cl. CPC A61M 5/31515; A61M 5/31583; A61M 2207/00

(57) ABSTRACT

The present invention is directed to a method for assembling a drug delivery device with a drive mechanism and a bung movably provided in a cartridge. The drive mechanism includes a piston rod and a bearing for driving the bung in a distal direction, wherein the bearing and the piston rod are configured to be connected via a snap connection and wherein the contact between the drive mechanism and the bung is indicated by a detectable signal produced by the engaging snap connection. The method includes the steps of placing the bearing on the bung, displacing the piston rod towards the bearing such that the snap connection is established, monitoring for the signal and stopping displacement of the piston rod upon detection of the signal. The invention is further directed to drug delivery device produced according to the respective method.

### CROSS REFERENCE TO RELATED APPLICATIONS

The present application is a U.S. National Phase Application pursuant to 35 U.S.C. §371 of International Application No. PCT/EP2013/067059 filed Aug. 15, 2013, which claims priority to European Patent Application No. 12180960.2 filed Aug. 20, 2012. The entire disclosure contents of these applications are herewith incorporated by reference into the present application.

### FIELD OF INVENTION

The present invention is directed at a method for assembling a drug delivery device with a drive mechanism and a bung movably provided in a cartridge, wherein the drive mechanism includes a piston rod and a bearing for driving the bung in a distal direction for delivering a medicament such as insulin. The invention is further directed at a drug delivery device produced by said method.

### BACKGROUND

Pen type drug delivery devices have applications where regular injection by persons without formal medical training occurs. This is increasingly common among patients having diabetes or the like. Self-treatment enables such patients to conduct effective management of their disease. The injection pens usually comprise a housing in which the drive mechanism is located. Some kinds of drug delivery devices also comprise a compartment to accommodate a cartridge in which the medicament is received. With the drive mechanism, the bung in the cartridge is displaced for dispensing the medicament accommodated therein. The drive mechanism includes a piston rod that has a bearing at one end, wherein the bearing is arranged in such manner such that it faces the bung. With the piston rod, the bearing is displaced toward the bung and urges the bung toward a distal end (needle end) of the drug delivery device, which is closest to the dispensing end of the device. Medicament from the cartridge is dispensed thereby. The opposite side of the device is referred to as the proximal end.

In devices of the generic kind, the manufacture may bring unavoidable tolerances and functional clearances between the single components of the drug delivery device, in particular the drive mechanism. As a consequence, clearances such as a gap between the elements of the drive mechanism such as between the bearing and the cartridge bung, may occur even after the drug delivery device has been assembled so that the bung may not be in contact with the distal end of the bearing. It is, therefore, important for a user to eliminate the gap between the cartridge bung and the distal end of the bearing and to bring the drive mechanism in a prestressed state prior to use. Otherwise, it would be possible that the dialed dose may not be dispensed from the device correctly. Initial clearances may already falsify the setting of the dose. To adjust the drug delivery device for use, priming actions are conducted to ensure that the drive mechanism is correctly adjusted, e.g. that the drive mechanism is in contact with the bung so that the correct amount of the medicament can expelled from the device. These actions often come along with a small amount of medicament being dispensed which gives a visual indication that the drug delivery device is ready to use.

It is known in the art to conduct adjustment of the drug delivery device by measurement of the bearing and the bung position before pressing, resp. assembly. The parts are then adjusted according to the measured value such that the bearing is brought into contact with the bung. However, the assembly machines for this method are expensive and the required time cycle is very long.

In WO 2011/039229 A1, a gap between the end of a piston rod and a bung of a cartridge is adjusted by determining a contact-making between a bearing attached to a lead screw and the bung. In a first step, there is a gap between the bung and the bearing so that the bung and the bearing do not make contact. Then the lead screw is advanced until the bearing contacts the bung. This contact is detected by an increase in torque feedback. However, this method for detecting the contact may include only a comparatively small and slow increase in the force feedback, when the contact has been made, whereby the moment of the establishment of the contact is not clearly determinable. A comparable approach is followed in WO 2005/018721 A1.

WO 2012/017035 A1 describes a method for assembling a cartridge unit for a drug delivery device. A cartridge unit is connectable to a drive unit via a snap connection and includes a bung and a bearing-like drive part to facilitate interaction between the bung and a piston rod of a drive unit. The drive part and the piston rod are coupled via a snap connection when the cartridge unit is assembled to the drive unit. In a final assembly step, the cartridge unit is displaced towards the drive unit. The drive unit comprises a deformable member immovably located in the housing and arranged around the piston rod, wherein the deformable member is deformed when the cartridge unit is displaced towards the drive unit so that tolerance gaps are eliminated.

A different approach is described in U.S. 2011/0245780 A1, where an adjusting member is provided between a piston rod and a piston to allow adjusting the overall length of the piston rod to eliminate tolerance gaps. The adjusting member and the piston rod form a piston rod assembly, which is variable in length to modify any axial gap between the piston rod assembly and the piston to a predefined gap size, e.g. to zero so that the piston rod assembly and the piston mutually abut when the drug delivery device is assembled.

### SUMMARY

It is an object of the present invention to provide an improved drug delivery device. Further objective is to simplify the assembly and adjustment process of a drug delivery device. This is achieved by a method as defined in claim 1 and by a drug delivery device as defined in claim 7.

The present invention is based on the idea to detect a signal indicative for the contact between the drive mechanism and the bung in the cartridge in an efficient way. The bearing and the piston rod are each configured such as to be connected to each other via a snap connection and that the contact between the drive mechanism and the bung is indicated by a measurable signal such as a peak in force and/or torque and/or an audible feedback with either of the effects being produced by the snap connection i.e. in the moment, the bearing and the piston are coupled to each other and the snap connection engages. Consequently, the method includes the steps of placing the bearing on the bung and displacing the piston rod in direction of the bearing such that the piston rod will be coupled to the bearing upon further movement. A signal, like a force and/or torque feedback of the drive mechanism and/or an audible feedback of the engaging snap connection is monitored.

In the moment, the bearing touches the bung, respectively the connection via the snapping mechanism is established, the pattern of the force and/or the torque and/or the audible feedback will change due to a change in the reaction forces. The moment of the contact or the snapping of the snap connection thus becomes measurable.

In other words, the snap connection is configured such that the engaging snap connection produces a detectable feedback signal. Prior to the production of the feedback signal, the snap connection is not engaged and the bearing is not coupled to the piston rod. The assembly process, in particular during the movement of the piston rod relative to the casing to make contact resp. to snap engage with the bearing may be monitored or scanned for said feedback signal.

As for example, the result of establishing the snap connection may comprise of an audible click and/or measureable increase in the torque and/or the force feedback of the drive mechanism, which may each be characterized by a peak of the feedback signal. The peak can be defined as, for example, a threshold value or a sudden change in the gradient of the force, the torque and/or or the audible feedback pattern over time. As an alternative, the force or torque may be measured over a displacement length e.g. of a tool. A snap connection will produce a sudden change in force or torque feedback once the initial resistance of the snap connection is overcome, the sudden change can be measured and used to indicated the moment, the snap connection is established. At the same time, the snapping action of the connection produces an audible click sound which can also be detected by a suitable measuring means or by the human ear. Thus, the moment, the drive mechanism is connected to the bung can be clearly determined.

Reaching the peak or any other characterizing indicator which corresponds to the snapping action or the contact of the piston rod with the bearing may serve as a trigger to stop displacement of the piston rod. Upon detection of the signal produced by the engaging snap connection, the displacement of the piston rod is stopped. The moment the feedback signal is produced corresponds to the achieved coupling between piston rod and bearing, which is taken as the occasion to stop the displacement of the piston rod. The underlying method may not only be used to reliably indicate a contact between the piston rod and the bearing but may also serve to trigger the stopping of the displacement of the piston rod, which may be characterized as a feedback-controlled-displacement of the piston rod, wherein the feedback signal to trigger the stopping of the piston rod is produced by the engaging snap connection.

According to another embodiment of the invention, the drive mechanism is accommodated in a casing and the cartridge is housed in a cartridge holder. The method includes the steps of placing the bearing on the bung in a first step, attaching the cartridge holder with the bung and the bearing thereon to the casing in a following step, displacing the piston rod towards the bearing such that the snap connection is established. The signal produced by the engaging snap connection is monitored and the displacement of the piston rod is stopped upon detection of the signal.

The method underlying the invention may be carried out such that the bearing is placed on the bung in a first step, preferably such that the bearing makes contact with the bung. Then, in a second step, the cartridge holder with the bearing and the bung maybe attached to the casing which houses the drive mechanism. Preferably, the attachment process is such that the bearing already approaches the piston rod but does not make contact with the piston rod, yet. However, the basic idea of the invention is not left when the piston rod just touches the bearing during the attachment of the cartridge holder to the housing but the snap connection is not being established during the attachment of the cartridge holder to the casing. It has proven effective that the displacement of the piston rod relative to the casing leads to the engagement of the snap connection, wherein the casing preferably remains stationary relative to the cartridge holder.

In the third step, after accomplishing the second step, the piston rod is displaced towards the bung resp. the bearing and relative to the casing, which leads to the engagement of the snap connection, it may scanned for a characteristic signal feedback. As explained above, this may be a sudden change in a force and/or torque feedback. During the displacement, the force and/or the torque required to displace the piston toward the bung may either be measured with suitable measurement systems or with haptical detection by a user. When the snap connection engages, a torque/force pattern suddenly changes due to the feedback snapping characteristics. This sudden signal change constitutes a feedback trigger signal for stopping the displacement of the piston. In other words, suddenly changing force and/or toque and/or audible feedbacks, which significantly differ from the feedback that is received before the snap connection is not engaged, signalize the establishment of the snap connection. The same applies to a produced click sound, which representatively illustrates that not even a change in a feedback signal pattern is necessary to indicate the engaging snap connection but that the production of a measureable feedback signal by the engaging snap connection (e.g. a characteristic audible click sound) is adequate to signalize the engagement between piston rod and bearing.

In comparison to other detecting methods, such as pure contact-measurement, the snap connection is characterized by an intense change in a signal pattern of a monitored signal, such as a striking feedback signal, or by a sudden emerging feedback reaction of the snapping connection. The intense feedback when the bearing and the piston rod snap together produces a change in a signal feedback that is clearly distinguishable from the signal pattern before the snap connection is established. One major advantage of the method is that the change in the signal pattern resp. a feedback signal is clearly and immediately determinable. In particular, the produced peak is clearly distinguishable from the signal noise of measurement systems, which makes the described method more reliable in comparison to known procedures.

By these measures, the drug delivery device may be prepared in an optimal prestressed condition right after manufacturing and further priming actions are dispensable.

The piston rod may be displaced by the drive mechanism, which may be configured such that by operating the drive mechanism, the piston rod is displaced relative to the casing in distal and/or proximal direction. To stop the displacement of the piston rod, operation of the drive mechanism may be interrupted resp. stopped.

In order to provide for a convenient assembly, a cartridge holder housing a cartridge may be attached to a casing accommodating the drive mechanism before displacing the piston rod. Especially in regard of disposable pens, this provides for enhanced reliability regarding the adjustment of the drive mechanism with respect to the bung.

Preferably, the piston rod is displaced in distal direction along a helical path with a rotary component and the translational component. The piston rod may be a lead screw, which can be in threaded engagement with a body. Advantageously, the body is at least partly surrounding the lead screw. The position of the piston rod relative to the bung may be adjusted by applying torque to the lead screw or to the body.

For providing an easy adjustment process during assembly and to reduce the complexity of the adjustment and assembly processes, the monitoring force and/or torque feedback is carried out at a dose setting mechanism. Accordingly, no expensive means are necessary to connect the feedback signal to a measurement device.

The object of the present invention is further achieved by a drug delivery device which is produced according to any of the methods described herein.

It is preferred, when the cartridge of the drug delivery device is filled with the medicament during assembly of the device. Also, the drug delivery device can be a disposable injection device. Such devices can be thrown away or recycled after the content of the medicament has been exhausted. However, the present invention is also applicable with re-usable devices designed to replace an emptied cartridge with a filled one aft er the whole content of the former cartridge has been administered.

An example of a disposable device in which the present invention may be used is given in EP 1 974 761 A2.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(w-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while α and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE DRAWINGS

In the following, the invention will be described by way of an example and with reference to the schematic drawings in which:

FIG. 1 shows a perspective view of a cartridge bung and a drive mechanism in a start position before adjustment;

FIG. 2 shows a schematic view of the elements shown in FIG. 1 in an end position after adjustment; and

FIG. 3 shows the pattern of force and torque over time during an adjustment process.

### DETAILED DESCRIPTION

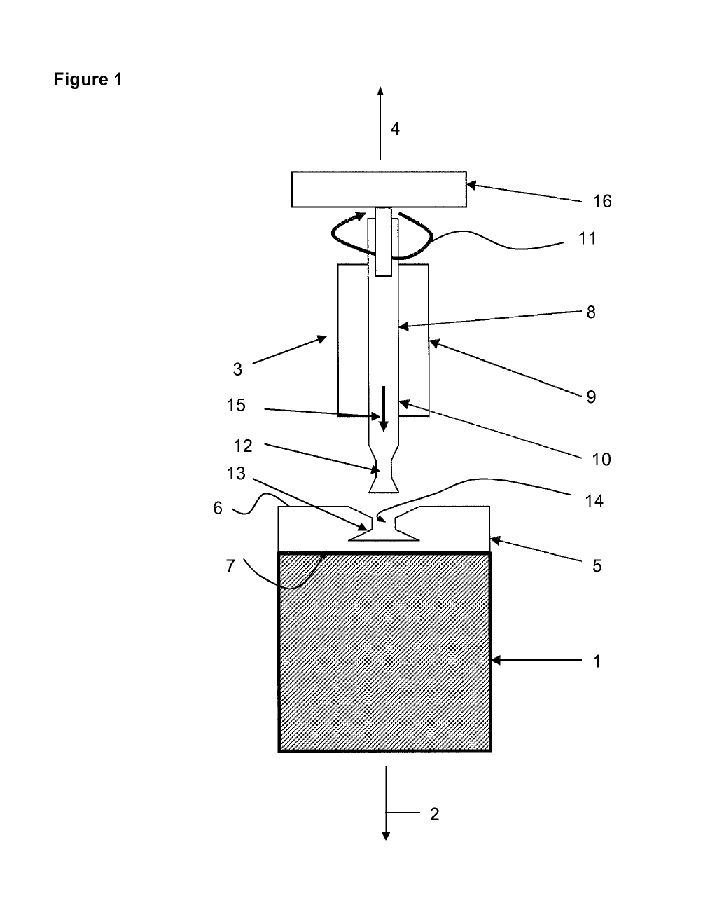

FIG. 1 shows a cartridge bung 1 for expelling a medicament out of a cartridge (not shown) in a distal direction 2. A distal movement of the cartridge bung is induced by a drive mechanism 3 located in proximal direction 4 from the cartridge bung 1. The drive mechanism 3 comprises a bearing 5 with a proximal end surface 6. A distal end surface of the bearing lies on and abuts a proximal end surface 7 of the cartridge bung 1. The cartridge (not shown) is housed in a cartridge holder (not shown) and the drive mechanism 3 is accommodated in a casing (not shown). The cartridge holder is attachable to the casing, wherein the bearing 5 is placed on the bung 1 before the cartridge holder is attached to the casing.

During manufacture of a drug delivery device, in particular, when the cartridge holder is attached to the casing, a piston rod or lead screw 8 of elongated shape is arranged spaced apart from the bearing 5 in proximal direction 4 as shown in FIG. 1. The lead screw 8 is arranged to be connected to the bearing 5 in such manner, that a movement of the lead screw 8 in distal direction moves the bearing 5 in the same. During this movement, the lead screw 8 also moves relative to the casing (now shown).

A proximal section of the lead screw 8 is surrounded by a body 9, wherein the lead screw 8 and the body 9 are connected to each other via a thread connection 10. The thread connection between the lead screw 8 and the body 9 is configured such that a relative rotation between the elements 8 and 9 results in a translational movement of the lead screw 8 relative to the body 9 in proximal or distal direction. As an example, by applying torque to the lead screw 8 in the direction indicated by arrow 11, the lead screw 8 screws through the body 9 in distal direction.

The distal end of the lead screw 8 is provided with a narrow section 12 or recess following the distal end of the lead screw 8 in proximal direction. The bearing 5 is on its proximal side 6 provided with a recess 13 or undercut which is adapted to accommodate the distal end of the lead screw 8, thus forming a snap-fit. The recess 13 is provided with an insertion section 14, which is narrower than the distal end of the lead screw 8. The narrow section 12 and the recess 13 are configured to establish a snap connection, which couples the bearing 5 to the lead screw 8.

By applying torque 11 to the lead screw 8, the lead screw 8 moves toward the recess 13 of the bearing 5 as indicated by the translational movement 15. This relative movement will continue until the lead screw 8 contacts the proximal surface of the bearing 5 such that the snap connection is established.

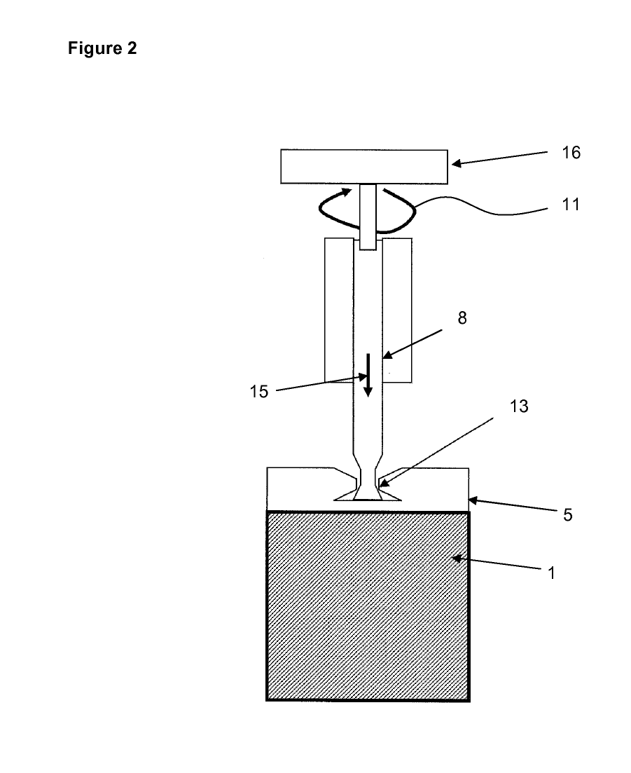

Further movement of the lead screw 8 forces the distal end of the lead screw 8 into the recess 13 of the bearing 5 as shown in FIG. 2. As the distal end of the lead screw 8 is wider than the recess 13 of the bearing 5, the snap connection emits an audible feedback in the form of a click. Another effect of the snap connection is, that when applying a constant torque and/or axial force to displace the lead screw 8 in axial direction a sudden change in the torque and/or the force feedback occurs as a higher resistance comes up, when the snap connection is being established. In the embodiment shown, the torque is measured via torque measuring means 16 provided at the proximal end of the drive mechanism 3.

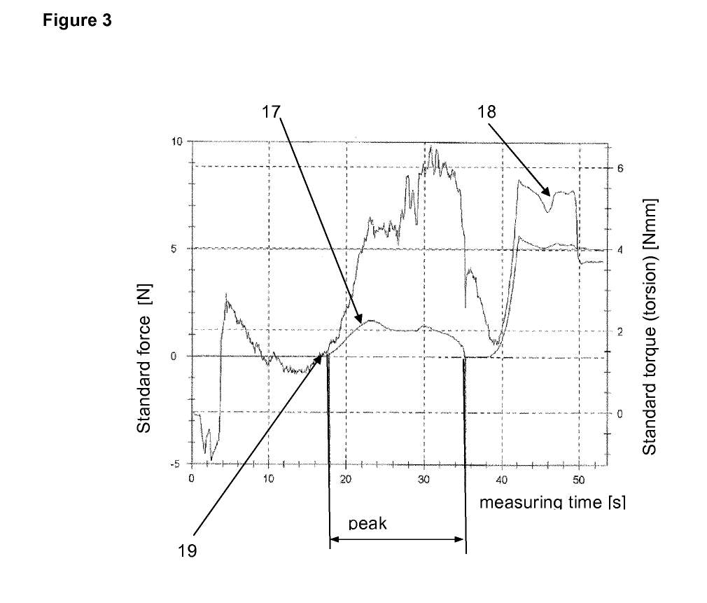

FIG. 3 displays a pattern of a counter force 17 respectively a counter torque 18 over time resulting from the driving force applied to the drive mechanism. As indicated by the arrow 19, which identifies the moment, the drive mechanism 3 is coupled to the bearing 5, a sudden change in the pattern of the respective graph can be recognized. The graphs (17, 18) each show a clear peak resulting from the sudden change of the counter force which is due to the contact-making with the bearing.

After the snap connection is established, the adjustment process is finished. Further displacement of the lead screw 8 can be stopped as the monitored feedback signals have indicated that the drug delivery device is in prestressed condition.

1-9. (canceled)

10. Method for assembling a drug delivery device with a drive mechanism and a bung movably provided in a cartridge, wherein said drive mechanism includes a piston rod and a bearing for driving the bung in a distal direction, wherein the bearing and the piston rod are configured to be connected via a snap connection and wherein the contact between the drive mechanism and the bung is indicated by a detectable signal produced by the engaging snap connection, said method including the steps of:
placing the bearing on the bung;
displacing the piston rod towards the bearing such that the snap connection is established and monitoring for the signal;
stopping displacement of the piston rod upon detection of the signal.

11. Method according to claim 10, characterized in that the signal is a peak in force and/or torque feedback and/or audible feedback.

12. Method according to claim 10, characterized in that monitoring the signal includes measuring the force or torque of a tool actuating a component of the drive mechanism.

13. Method according to claim 10, characterized in that the piston rod is displaced in distal direction along a helical path with a rotary component and a translational component.

14. Method according to claim 10, wherein the piston rod is a lead screw, which is threadedly engaged with a body, characterized in that the position of the lead screw relative to the bung is adjusted by applying torque to the lead screw or to the body.

15. Method according to claim 10, wherein the drive mechanism is accommodated in a casing and the cartridge is housed in a cartridge holder, wherein the cartridge holder with the cartridge is attached to the casing before the piston rod is displaced towards the bearing.

16. Drug delivery device produced according to claim 10.

17. Drug delivery device according to claim 16, characterized in that the cartridge is filled with a medicament.

18. Drug delivery device according to claim 16, characterized in that the drug delivery device is a disposable injection device.
